(19) United States
(12) Patent Application Publication
(10) Pub. No.: US 2026/0128134 A1
(43) Pub. Date: May 7, 2026

(54) Creation of Realistic MS/MS Spectra for Putative Designer Drugs

(71) Applicant: DH Technologies Development Pte. Ltd., Singapore (SG)

(72) Inventors: Lyle Lorrence BURTON, Woodbridge (CA); David Michael COX, Toronto (CA)

(21) Appl. No.: 19/118,904

(22) PCT Filed: Oct. 6, 2023
(86) PCT No.: PCT/IB2023/060028

§ 371 Date: Apr. 7, 2025

Related U.S. Application Data
(60) Provisional application No. 63/378,594, filed on Oct. 6, 2022.

(51) Int. Cl. G16C 20/20; G16C 20/30; G16C 20/60

(52) U.S. Cl. CPC G16C 20/20; G16C 20/30; G16C 20/60

(57) ABSTRACT

Systems and methods are provided for predicting the mass spectrum of an unknown compound. An experimental mass spectrum of a known compound is obtained. One or more mass peaks of the experimental mass spectrum corresponding to a substructure of the known compound are annotated with at least one modification an unknown compound is predicted to include. An in-silico mass spectrum is created for the unknown compound from the experimental mass spectrum and the annotated one or more mass peaks. The unknown compound is then identified from a sample by mass analyzing the sample, producing an unknown experimental mass spectrum, and comparing the unknown experimental mass spectrum to the in-silico mass spectrum.

### RELATED APPLICATIONS

This application claims the benefit of U.S. Provisional Patent Application Ser. No. 63/378,594, filed on Oct. 6, 2022, the content of which is incorporated by reference herein in its entirety.

### INTRODUCTION

The teachings herein relate to predicting the mass spectrum of an unknown compound. More particularly the teachings herein relate to systems and methods for annotating mass peaks of a mass spectrum of a known compound with at least one modification an unknown compound is predicted to include and creating an in-silico mass spectrum for the unknown compound from the experimental mass spectrum and the annotation.

The systems and methods herein can be performed in conjunction with a processor, controller, or computer system, such as the computer system of FIG. 1.

### Compound Identification Problem

Compound identification of unknown compounds is a very difficult problem. It is especially difficult when the compound is novel and has not been previously or widely described in the literature.

A scenario encountered, for example, in the forensics laboratory is the need to identify a compound believed to be potentially responsible for a fatality. A mass spectrometry/mass spectrometry (MS/MS) or product ion mass spectrum may be obtained for a sample believed to contain the compound. A suspected mass peak for the compound and its molecular weight may be obtained from the mass spectrum. However, the mass peak may not match to any known compound, substance, or, more specifically, to any known drug of abuse.

As a result, there is a need for additional systems and methods to predict the mass spectra of unknown compounds and add them to a library or database of mass spectra so that compounds such as “designer” drugs of abuse can be quickly and automatically identified by laboratory instruments.

### LC-MS and LC-MS/MS Background

Mass spectrometry (MS) is an analytical technique for the detection and quantitation of chemical compounds based on the analysis of mass-to-charge ratios (m/z) of ions formed from those compounds. The combination of mass spectrometry (MS) and liquid chromatography (LC) is an important analytical tool for the identification and quantitation of compounds within a mixture. Generally, in liquid chromatography, a fluid sample under analysis is passed through a column filled with a chemically-treated solid adsorbent material (typically in the form of small solid particles, e.g., silica). Due to slightly different interactions of components of the mixture with the solid adsorbent material (typically referred to as the stationary phase), the different components can have different transit (elution) times through the packed column, resulting in separation of the various components.

Note that the terms “mass” and “m/z” are used interchangeably herein. One of ordinary skill in the art understands that a mass can be found from an m/z by multiplying the m/z by the charge. Similarly, the m/z can be found from a mass by dividing the mass by the charge.

In LC-MS, the effluent exiting the LC column can be continuously subjected to MS analysis. The data from this analysis can be processed to generate an extracted ion chromatogram (XIC), which can depict detected ion intensity (a measure of the number of detected ions of one or more particular analytes) as a function of retention time.

In MS analysis, an MS or precursor ion scan is performed at each interval of the separation for a mass range that includes the precursor ion. An MS scan includes the selection of a precursor ion or precursor ion range and mass analysis of the precursor ion or precursor ion range.

In some cases, the LC effluent can be subjected to tandem mass spectrometry (or mass spectrometry/mass spectrometry MS/MS) for the identification of product ions corresponding to the peaks in the XIC. For example, the precursor ions can be selected based on their mass/charge ratio to be subjected to subsequent stages of mass analysis. For example, the selected precursor ions can be fragmented (e.g., via collision-induced dissociation), and the fragmented ions (product ions) can be analyzed via a subsequent stage of mass spectrometry.

### Fragmentation Techniques Background

Electron-based dissociation (ExD), ultraviolet photodissociation (UVPD), infrared photodissociation (IRMPD), and collision-induced dissociation (CID) are often used as fragmentation techniques for tandem mass spectrometry (MS/MS). CID is the most conventional technique for dissociation in tandem mass spectrometers.

ExD can include, but is not limited to, electron-induced dissociation (EID), electron impact excitation in organics (EIEIO), electron capture dissociation (ECD), or electron transfer dissociation (ETD).

### Tandem Mass Spectrometry or MS/MS Background

Tandem mass spectrometry or MS/MS involves ionization of one or more compounds of interest from a sample, selection of one or more precursor ions of the one or more compounds, fragmentation of the one or more precursor ions into product ions, and mass analysis of the product ions.

Tandem mass spectrometry can provide both qualitative and quantitative information. The product ion spectrum can be used to identify a molecule of interest. The intensity of one or more product ions can be used to quantitate the amount of the compound present in a sample.

A large number of different types of experimental methods or workflows can be performed using a tandem mass spectrometer. These workflows can include, but are not limited to, targeted acquisition, information dependent acquisition (IDA) or data dependent acquisition (DDA), and data independent acquisition (DIA).

In a targeted acquisition method, one or more transitions of a precursor ion to a product ion are predefined for a compound of interest. As a sample is being introduced into the tandem mass spectrometer, the one or more transitions are interrogated during each time period or cycle of a plurality of time periods or cycles. In other words, the mass spectrometer selects and fragments the precursor ion of each transition and performs a targeted mass analysis for the product ion of the transition. As a result, a chromatogram (the variation of the intensity with retention time) is produced for each transition. Targeted acquisition methods include, but are not limited to, multiple reaction monitoring (MRM) and selected reaction monitoring (SRM).

MRM experiments are typically performed using “low resolution” instruments that include, but are not limited to, triple quadrupole (QqQ) or quadrupole linear ion trap (QqLIT) devices. With the advent of “high resolution” instruments, there was a desire to collect MS and MS/MS using workflows that are similar to QqQ/QqLIT systems. High-resolution instruments include, but are not limited to, quadrupole time-of-flight (QqTOF) or orbitrap devices. These high-resolution instruments also provide new functionality.

MRM on QqQ/QqLIT systems is the standard mass spectrometric technique of choice for targeted quantification in all application areas, due to its ability to provide the highest specificity and sensitivity for the detection of specific components in complex mixtures. However, the speed and sensitivity of today's accurate mass systems have enabled a new quantification strategy with similar performance characteristics. In this strategy (termed MRM high resolution (MRM-HR) or parallel reaction monitoring (PRM)), looped MS/MS spectra are collected at high-resolution with short accumulation times, and then fragment ions (product ions) are extracted post-acquisition to generate MRM-like peaks for integration and quantification. With instrumentation like the TRIPLETOF® Systems of AB SCIEX™, this targeted technique is sensitive and fast enough to enable quantitative performance similar to higher-end triple quadrupole instruments, with full fragmentation data measured at high resolution and high mass accuracy.

In other words, in methods such as MRM-HR, a high-resolution precursor ion mass spectrum is obtained, one or more precursor ions are selected and fragmented, and a high-resolution full product ion spectrum is obtained for each selected precursor ion. A full product ion spectrum is collected for each selected precursor ion but a product ion mass of interest can be specified and everything other than the mass window of the product ion mass of interest can be discarded.

In an IDA (or DDA) method, a user can specify criteria for collecting mass spectra of product ions while a sample is being introduced into the tandem mass spectrometer. For example, in an IDA method a precursor ion or mass spectrometry (MS) survey scan is performed to generate a precursor ion peak list. The user can select criteria to filter the peak list for a subset of the precursor ions on the peak list. The survey scan and peak list are periodically refreshed or updated, and MS/MS is then performed on each precursor ion of the subset of precursor ions. A product ion spectrum is produced for each precursor ion. MS/MS is repeatedly performed on the precursor ions of the subset of precursor ions as the sample is being introduced into the tandem mass spectrometer.

In proteomics and many other applications, however, the complexity and dynamic range of compounds is very large. This poses challenges for traditional targeted and IDA methods, requiring very high-speed MS/MS acquisition to deeply interrogate the sample in order to both identify and quantify a broad range of analytes.

As a result, DIA methods, the third broad category of tandem mass spectrometry, were developed. These DIA methods have been used to increase the reproducibility and comprehensiveness of data collection from complex samples. DIA methods can also be called non-specific fragmentation methods. In a DIA method the actions of the tandem mass spectrometer are not varied among MS/MS scans based on data acquired in a previous precursor or survey scan. Instead, a precursor ion mass range is selected. A precursor ion mass selection window is then stepped across the precursor ion mass range. All precursor ions in the precursor ion mass selection window are fragmented and all of the product ions of all of the precursor ions in the precursor ion mass selection window are mass analyzed.

The precursor ion mass selection window used to scan the mass range can be narrow so that the likelihood of multiple precursors within the window is small. This type of DIA method is called, for example, MS/MS^{ALL}. In an MS/MS^{ALL} method, a precursor ion mass selection window of about 1 Da is scanned or stepped across an entire mass range. A product ion spectrum is produced for each 1 Da precursor mass window. The time it takes to analyze or scan the entire mass range once is referred to as one scan cycle. Scanning a narrow precursor ion mass selection window across a wide precursor ion mass range during each cycle, however, can take a long time and is not practical for some instruments and experiments.

As a result, a larger precursor ion mass selection window, or selection window with a greater width, is stepped across the entire precursor mass range. This type of DIA method is called, for example, SWATH acquisition. In a SWATH acquisition, the precursor ion mass selection window stepped across the precursor mass range in each cycle may have a width of 5-25 Da, or even larger. Like the MS/MS^{ALL} method, all of the precursor ions in each precursor ion mass selection window are fragmented, and all of the product ions of all of the precursor ions in each mass selection window are mass analyzed. However, because a wider precursor ion mass selection window is used, the cycle time can be significantly reduced in comparison to the cycle time of the MS/MS^{ALL} method.

U.S. Pat. No. 8,809,770 describes how SWATH acquisition can be used to provide quantitative and qualitative information about the precursor ions of compounds of interest. In particular, the product ions found from fragmenting a precursor ion mass selection window are compared to a database of known product ions of compounds of interest. In addition, ion traces or extracted ion chromatograms (XICs) of the product ions found from fragmenting a precursor ion mass selection window are analyzed to provide quantitative and qualitative information.

However, identifying compounds of interest in a sample analyzed using SWATH acquisition, for example, can be difficult. It can be difficult because either there is no precursor ion information provided with a precursor ion mass selection window to help determine the precursor ion that produces each product ion, or the precursor ion information provided is from a mass spectrometry (MS) observation that has a low sensitivity. In addition, because there is little or no specific precursor ion information provided with a precursor ion mass selection window, it is also difficult to determine if a product ion is convolved with or includes contributions from multiple precursor ions within the precursor ion mass selection window.

As a result, a method of scanning the precursor ion mass selection windows in SWATH acquisition, called scanning SWATH, was developed. Essentially, in scanning SWATH, a precursor ion mass selection window is scanned across a mass range so that successive windows have large areas of overlap and small areas of non-overlap. This scanning makes the resulting product ions a function of the scanned precursor ion mass selection windows. This additional information, in turn, can be used to identify the one or more precursor ions responsible for each product ion.

Scanning SWATH has been described in International Publication No. WO 2013/171459 A2 (hereinafter “the '459 Application”). In the '459 Application, a precursor ion mass selection window or precursor ion mass selection window of 25 Da is scanned with time such that the range of the precursor ion mass selection window changes with time. The timing at which product ions are detected is then correlated to the timing of the precursor ion mass selection window in which their precursor ions were transmitted.

The correlation is done by first plotting the mass-to-charge ratio (m/z) of each product ion detected as a function of the precursor ion m/z values transmitted by the quadrupole mass filter. Since the precursor ion mass selection window is scanned over time, the precursor ion m/z values transmitted by the quadrupole mass filter can also be thought of as times. The start and end times at which a particular product ion is detected are correlated to the start and end times at which its precursor is transmitted from the quadrupole. As a result, the start and end times of the product ion signals are used to determine the start and end times of their corresponding precursor ions.

### SUMMARY

A method and computer program product are disclosed for predicting the mass spectrum of an unknown compound. An experimental mass spectrum of a known compound is obtained. One or more mass peaks of the experimental mass spectrum corresponding to a substructure of the known compound are annotated with at least one modification an unknown compound is predicted to include. Finally, an in-silico mass spectrum is created for the unknown compound from the experimental mass spectrum and the annotated one or more mass peaks.

A system is disclosed for identifying an unknown compound that includes a processor. The processor obtains an experimental mass spectrum of a known compound. The processor annotates one or more mass peaks of the experimental mass spectrum corresponding to a substructure of the known compound with at least one modification an unknown compound is predicted to include. The processor creates an in-silico mass spectrum for the unknown compound from the experimental mass spectrum and the annotated one or more mass peaks. The processor obtains an unknown experimental mass spectrum. Finally, the processor determines that the unknown compound is a modification of the known compound if the unknown experimental mass spectrum matches the in-silico mass spectrum. In some embodiments, the system can further comprise a mass spectrometer and wherein the processor instructs the mass spectrometer to obtain the unknown experimental mass spectrum by analyzing the unknown compound.

These and other features of the applicant's teachings are set forth herein.

### BRIEF DESCRIPTION OF THE DRAWINGS

The skilled artisan will understand that the drawings, described below, are for illustration purposes only. The drawings are not intended to limit the scope of the present teachings in any way.

FIG. 1 is a block diagram that illustrates a computer system, upon which embodiments of the present teachings may be implemented.

FIG. 2 is a schematic diagram of a system for identifying an unknown compound, in accordance with various embodiments.

FIG. 3 is an exemplary flowchart showing a method for predicting the mass spectrum of an unknown compound, in accordance with various embodiments.

FIG. 4 is a schematic diagram of a system that includes one or more distinct software modules and that performs a method for predicting the mass spectrum of an unknown compound, in accordance with various embodiments.

Before one or more embodiments of the present teachings are described in detail, one skilled in the art will appreciate that the present teachings are not limited in their application to the details of construction, the arrangements of components, and the arrangement of steps set forth in the following detailed description or illustrated in the drawings. Also, it is to be understood that the phraseology and terminology used herein is for the purpose of description and should not be regarded as limiting.

### DESCRIPTION OF VARIOUS EMBODIMENTS

### Computer-Implemented System

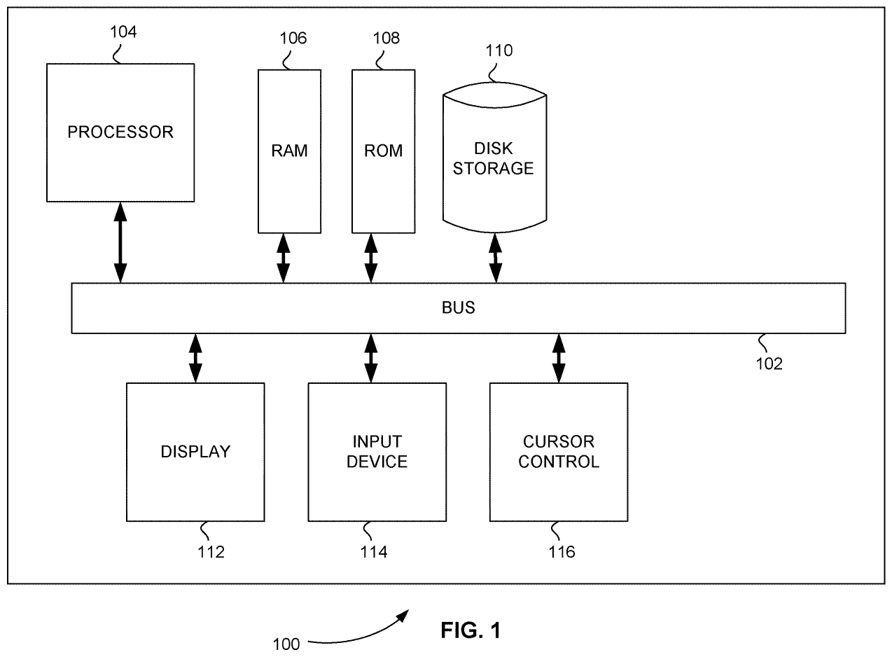

FIG. 1 is a block diagram that illustrates a computer system 100, upon which embodiments of the present teachings may be implemented. Computer system 100 includes a bus 102 or other communication mechanism for communicating information, and a processor 104 coupled with bus 102 for processing information. Computer system 100 also includes a memory 106, which can be a random-access memory (RAM) or other dynamic storage device, coupled to bus 102 for storing instructions to be executed by processor 104. Memory 106 also may be used for storing temporary variables or other intermediate information during execution of instructions to be executed by processor 104. Computer system 100 further includes a read only memory (ROM) 108 or other static storage device coupled to bus 102 for storing static information and instructions for processor 104. A storage device 110, such as a magnetic disk or optical disk, is provided and coupled to bus 102 for storing information and instructions.

Computer system 100 may be coupled via bus 102 to a display 112, such as a cathode ray tube (CRT) or liquid crystal display (LCD), for displaying information to a computer user. An input device 114, including alphanumeric and other keys, is coupled to bus 102 for communicating information and command selections to processor 104. Another type of user input device is cursor control 116, such as a mouse, a trackball or cursor direction keys for communicating direction information and command selections to processor 104 and for controlling cursor movement on display 112.

A computer system 100 can perform the present teachings. Consistent with certain implementations of the present teachings, results are provided by computer system 100 in response to processor 104 executing one or more sequences of one or more instructions contained in memory 106. Such instructions may be read into memory 106 from another computer-readable medium, such as storage device 110. Execution of the sequences of instructions contained in memory 106 causes processor 104 to perform the process described herein.

Alternatively, hard-wired circuitry may be used in place of or in combination with software instructions to implement the present teachings. For example, the present teachings may also be implemented with programmable artificial intelligence (AI) chips with only the encoder neural network programmed—to allow for performance and decreased cost. Thus, implementations of the present teachings are not limited to any specific combination of hardware circuitry and software.

The term “computer-readable medium” or “computer program product” as used herein refers to any media that participates in providing instructions to processor 104 for execution. The terms “computer-readable medium” and “computer program product” are used interchangeably throughout this written description. Such a medium may take many forms, including but not limited to, non-volatile media and volatile media. Non-volatile media includes, for example, optical or magnetic disks, such as storage device 110. Volatile media includes dynamic memory, such as memory 106.

Common forms of computer-readable media include, for example, a floppy disk, a flexible disk, hard disk, magnetic tape, or any other magnetic medium, a CD-ROM, digital video disc (DVD), a Blu-ray Disc, any other optical medium, a thumb drive, a memory card, a RAM, PROM, and EPROM, a FLASH-EPROM, any other memory chip or cartridge, or any other tangible medium from which a computer can read.

Various forms of computer readable media may be involved in carrying one or more sequences of one or more instructions to processor 104 for execution. For example, the instructions may initially be carried on the magnetic disk of a remote computer. The remote computer can load the instructions into its dynamic memory and send the instructions over a telephone line using a modem. A modem local to computer system 100 can receive the data on the telephone line and use an infra-red transmitter to convert the data to an infra-red signal. An infra-red detector coupled to bus 102 can receive the data carried in the infra-red signal and place the data on bus 102. Bus 102 carries the data to memory 106, from which processor 104 retrieves and executes the instructions. The instructions received by memory 106 may optionally be stored on storage device 110 either before or after execution by processor 104.

In accordance with various embodiments, instructions configured to be executed by a processor to perform a method are stored on a computer-readable medium. The computer-readable medium can be a device that stores digital information. For example, a computer-readable medium includes a compact disc read-only memory (CD-ROM) as is known in the art for storing software. The computer-readable medium is accessed by a processor suitable for executing instructions configured to be executed.

The following descriptions of various implementations of the present teachings have been presented for purposes of illustration and description. It is not exhaustive and does not limit the present teachings to the precise form disclosed. Modifications and variations are possible in light of the above teachings or may be acquired from practicing of the present teachings. Additionally, the described implementation includes software but the present teachings may be implemented as a combination of hardware and software or in hardware alone. The present teachings may be implemented with both object-oriented and non-object-oriented programming systems.

### Spectrum Prediction Using Annotated Modifications

As described above, compound identification of unknown compounds is a very difficult problem. It is especially difficult when the compound is novel and has not been previously or widely described in the literature.

A scenario encountered, for example, in the forensics laboratory is the need to identify a “designer” drug believed to be potentially responsible for a fatality. A mass peak of a product ion mass spectrum obtained for the drug, however, may not match any known drug of abuse.

As a result, there is a need for additional systems and methods to predict the mass spectra of unknown compounds and add them to a library or database of mass spectra so that compounds such as “designer” drugs of abuse can be quickly and automatically identified by laboratory instruments.

Conventionally, one method of identifying designer drugs has been to modify the chemical structures of known drugs of abuse and then use an in-silico method to generate a predicted spectrum for each modification. Specifically, a collection of chemical structures of known drugs of abuse is created. Each structure is subjected to common “designer” modifications (e.g., addition of a methyl group) at chemically likely locations. At least algorithmically, this procedure is similar to in-silico drug metabolite predictions.

At this point, there is a collection of structures for the multiple known starting drugs for each of the potential modifications, For each of these structures, an in-silico MS/MS mass spectrum is generated using a software program. For example, one software program uses a database of various fragmentation rules to predict likely fragments (or product ions). Finally, the predicted spectra are added to an MS/MS library and a standard library search is performed using the spectrum of the unknown compound. Unknown compounds with a matching molecular weight and a tolerable library search score are identified as possible designer variations of the corresponding known drug.

In this method, the chemical structures and hence m/z of these fragments should be accurate. However, the intensities in the generated spectra cannot usually be predicted with any substantial degree of accuracy.

In various embodiments, an improved method relies on the assumption that the MS/MS fragment mass spectrum of the designer variant is likely to be substantially similar to that of the starting drug. In other words, m/z peaks corresponding to the substructure without the modification should not shift and those with the modification should shift to higher m/z by the mass of the modification. In both cases, the relative peak intensity is assumed to be approximately unchanged.

In this method, the first step is to annotate the experimental spectrum of each unmodified known drug. For example, each fragment m/z of the experimental spectrum is assigned to the corresponding sub-structure of the known drug. This can be done automatically using existing software tools, such as those developed for the quality control of compound libraries.

In a second step, an in-silico MS/MS spectrum is created for each variant by starting with the experimental spectrum of the known drug and shifting certain fragments in mass according to the modification suspected. As described above, for example, m/z peaks corresponding to a substructure without the annotated modification are not shifted in mass. However, those m/z peaks with the modification are shifted to a higher m/z by the mass of the modification. One ordinary skill in the art understands that synonyms for the term “in-silico” can include, but are not limited to, predicted, theoretical, or computer-generated.

For each designer variant (that includes one or modifications), the net result is an in-silico MS/MS spectrum. This spectrum is likely to be more accurate than previous methods obtained from purely theoretical predictions based on the structure. As described above, these purely theoretical predictions from the structure cannot usually predict intensities with any substantial degree of accuracy.

### System for Identifying an Unknown Compound

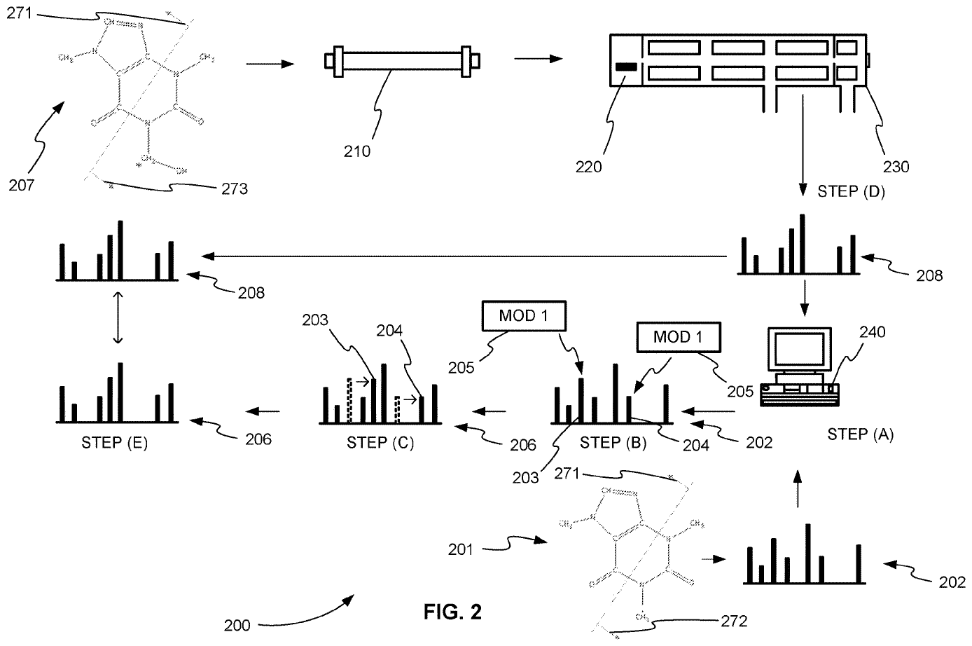

FIG. 2 is a schematic diagram 200 of a system for identifying an unknown compound, in accordance with various embodiments. The system includes mass spectrometer 230 and processor 240. Processor 240 can be, but is not limited to, a controller, a computer, a microprocessor, the computer system of FIG. 1, or any device capable of analyzing data. Processor 240 can also be any device capable of sending and receiving control signals and data.

In step (A), processor 240 obtains experimental mass spectrum 202 of known compound 201. Experimental mass spectrum 202 is obtained from a library of known spectra or measured from known compound 201, for example.

In various embodiments, for example, known compound 201 of FIG. 2 includes two typical fragments 271 and 272 for a small molecule that has broken between the two indicated C—N bonds. Fragment 271 is, for example, about a 109.1 Da fragment, and fragment 272 is about an 87.1 Da fragment (the indicated m/z assumes that one new bond is formed and that the fragments are protonated).

In step (B), processor 240 annotates one or more mass peaks of experimental mass spectrum 202 corresponding to a substructure of known compound 201 with at least one modification 205 unknown compound 207 is predicted to include. For example, in FIG. 2, peak 203 and peak 204 of experimental mass spectrum 202 are annotated with the same modification 205. In various embodiments (not shown), more than one modification can be annotated.

In step (C), processor 240 creates in-silico mass spectrum 206 for unknown compound 207 from experimental mass spectrum 202 and the annotated one or more mass peaks. For example, as shown in FIG. 2, processor 240 can shift peak 203 and peak 204 of experimental mass spectrum 202 by the m/z of modification 205 to create in-silico mass spectrum 206.

In step (D), processor 240 obtains an unknown experimental mass spectrum 208 of unknown compound 207. This can be received/obtained from mass spectrometer 230 or can be received from another system, computer or data store device in which the previously obtained unknown experimental mass spectrometer may be stored which can include random-access memory (RAM) or other dynamic storage device, read only memory (ROM) or other static storage device or storage device, such as a magnetic disk or optical disk.

In various embodiments, unknown compound 207 of FIG. 2 is a possible modification of known compound 201. Unknown compound 207 includes, for example, two fragments 271 and 273. Fragment 271 is the same fragment as shown in known compound 201. Fragment 273, however, contains a modification in comparison to fragment 272 and is about a 103.1 Da fragment. In this case, there is an addition of an oxygen at the ‘*’ carbon location in fragment 273. So, compared to fragment 272 of known compound 201, fragment 273 of unknown compound 207 is shifted by the modification mass difference (+16 in this case for oxygen).

In step (E), processor 240 determines if unknown experimental mass spectrum 208 matches in-silico mass spectrum 206. In various embodiments, determining if an unknown experimental mass spectrum matches an in-silico mass spectrum includes using a high purity or fit score from a standard library search algorithm. For example, a purity or fit score for the comparison of the spectra above a certain threshold level indicates a match.

In various embodiments, processor 240 further adds in-silico mass spectrum 206 to a mass spectrum library or database (not shown). This is a library of known compounds that can now be used to identify previously unknown compound 207.

In various embodiments, as shown in FIG. 2, processor 240 creates in-silico mass spectrum 206 by shifting an m/z of the one or more annotated mass peaks of experimental mass spectrum 202 according to at least one modification 205.

In various embodiments, as shown in FIG. 2, the one or more annotated mass peaks of experimental mass spectrum 202 are shifted to a higher m/z value in in-silico mass spectrum 206 according to at least one modification 205.

In various embodiments, as shown in FIG. 2, intensities of the shifted one or more annotated mass peaks of in-silico mass spectrum 206 are not changed from intensities of corresponding mass peaks of experimental mass spectrum 202.

In various embodiments, experimental mass spectrum 202, in-silico mass spectrum 206, and unknown experimental mass spectrum 208 are product ion spectra. In various alternative embodiments, experimental mass spectrum 202, in-silico mass spectrum 206, and unknown experimental mass spectrum 208 are precursor ion spectra.

In various embodiments, known compound 201 is a known drug of abuse and unknown compound 207 is a variant of the known drug of abuse.

In various embodiments, mass spectrometer 230 measures mass spectrum 208 and sends mass spectrum 208 to processor 240. Ion source device 220 of mass spectrometer 230 ionizes separated fragments of compound 207 or only compound 207, producing an ion beam. Ion source device 220 is controlled by processor 240, for example. Ion source device 220 is shown as a component of mass spectrometer 230. In various alternative embodiments, ion source device 220 is a separate device. Ion source device 220 can be, but is not limited to, an electrospray ion source (ESI) device or a chemical ionization (CI) source device, such as an atmospheric pressure chemical ionization source (APCI) device or an atmospheric pressure photoionization (APPI) source device.

Mass spectrometer 230 mass analyzes precursor ions of compound 207 or selects and fragments compound 207 and mass analyzes product ions of compound 207 from the ion beam at one or more different times. Mass spectrum 208 is produced for compound 207. Mass spectrometer 230 is controlled by processor 240, for example.

In the system of FIG. 2, mass spectrometer 230 is shown as a triple quadrupole device. One of ordinary skill in the art can appreciate that any component of mass spectrometer 230 can include other types of mass spectrometry devices including, but not limited to, ion traps, orbitraps, time-of-flight (TOF) devices, ion mobility devices, or Fourier transform ion cyclotron resonance (FT-ICR) devices.

In various embodiments, the system of FIG. 2 further includes additional device 210 that affects compound 201 before mass analysis, providing an additional dimension. As shown in FIG. 2, additional device 210 is an LC device and the at least one additional dimension or spectral data provided is retention time. In various alternative embodiments, additional device 210 can be, but is not limited to, a gas chromatography (GC) device, capillary electrophoresis (CE) device, an ion mobility spectrometry (IMS) device, or a differential mobility spectrometry (DMS) device.

### Method for Predicting the Mass Spectrum of an Unknown Compound

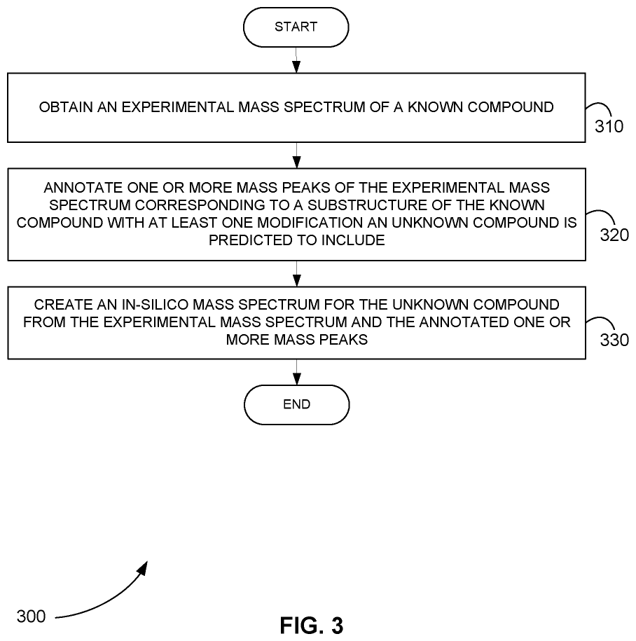

FIG. 3 is an exemplary flowchart showing a method 300 for predicting the mass spectrum of an unknown compound, in accordance with various embodiments.

In step 310 of method 800, an experimental mass spectrum of a known compound is obtained.

In step 320, one or more mass peaks of the experimental mass spectrum corresponding to a substructure of the known compound are annotated with at least one modification an unknown compound is predicted to include.

In step 330, create an in-silico mass spectrum for the unknown compound from the experimental mass spectrum and the annotated one or more mass peaks.

### Computer Program Product for Predicting the Mass Spectrum

In various embodiments, a computer program product includes a non-transitory tangible computer-readable storage medium whose contents include a program with instructions being executed on a processor so as to perform a method for predicting the mass spectrum of an unknown compound. This method is performed by a system that includes one or more distinct software modules.

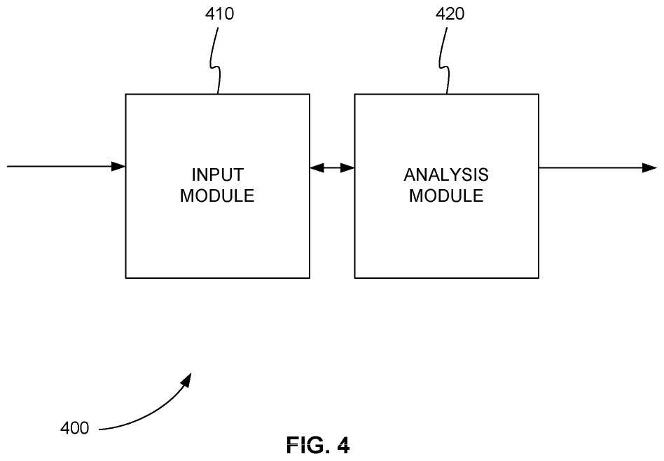

FIG. 4 is a schematic diagram of a system 400 that includes one or more distinct software modules and that performs a method for predicting the mass spectrum of an unknown compound, in accordance with various embodiments. System 400 includes input module 410 and analysis module 420.

Input module 410 obtains an experimental mass spectrum of a known compound. Analysis module 420 annotates one or more mass peaks of the experimental mass spectrum corresponding to a substructure of the known compound with at least one modification an unknown compound is predicted to include. Analysis module 420 creates an in-silico mass spectrum for the unknown compound from the experimental mass spectrum and the annotated one or more mass peaks.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

Further, in describing various embodiments, the specification may have presented a method and/or process as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process should not be limited to the performance of their steps in the order written, and one skilled in the art can readily appreciate that the sequences may be varied and still remain within the spirit and scope of the various embodiments.

What is claimed is:

1. A method for predicting the mass spectrum of an unknown compound, comprising:
(a) obtaining an experimental mass spectrum of a known compound;
(b) annotating one or more mass peaks of the experimental mass spectrum corresponding to a substructure of the known compound with at least one modification an unknown compound is predicted to include; and
(c) creating an in-silico mass spectrum for the unknown compound from the experimental mass spectrum and the annotated one or more mass peaks.

2. The method of any combination of claim 1, further comprising adding the in-silico mass spectrum to a mass spectrum library.

3. The method of any combination of claim 1, wherein the in-silico mass spectrum is created by shifting a mass-to-charge ratio (m/z) of the one or more annotated mass peaks of the experimental mass spectrum according to the at least one modification.

4. The method of claim 1, wherein the one or more annotated mass peaks of the experimental mass spectrum are shifted to a higher m/z value in the in-silico mass spectrum according to the at least one modification.

5. The method of claim 4, wherein intensities of the shifted one or more annotated mass peaks of the in-silico mass spectrum are not changed from intensities of corresponding mass peaks of the experimental mass spectrum.

6. The method of claim 1, wherein the experimental mass spectrum, the in-silico mass spectrum, and the unknown experimental mass spectrum are product ion spectra.

7. The method of claim 1, wherein the known compound comprises a known drug of abuse and the unknown compound comprises a variant of the known drug of abuse.

8. A computer program product, comprising a non-transitory tangible computer-readable storage medium whose contents cause a processor to perform a method for predicting the mass spectrum of an unknown compound, comprising:
providing a system, wherein the system comprises one or more distinct software modules, and wherein the distinct software modules comprise an input module and an analysis module;
obtaining an experimental mass spectrum of a known compound using the input module;
annotating one or more mass peaks of the experimental mass spectrum corresponding to a substructure of the known compound with at least one modification an unknown compound is predicted to include using the analysis module; and
creating an in-silico mass spectrum for the unknown compound from the experimental mass spectrum and the annotated one or more mass peaks using the analysis module.

9. The computer program product of claim 8, further comprising adding the in-silico mass spectrum to a mass spectrum library.

10. The computer program product of claim 8, wherein the in-silico mass spectrum is created by shifting a mass-to-charge ratio (m/z) of the one or more annotated mass peaks of the experimental mass spectrum according to the at least one modification.

11. The computer program product of claim 8, wherein the one or more annotated mass peaks of the experimental mass spectrum are shifted to a higher m/z value in the in-silico mass spectrum according to the at least one modification.

12. The computer program product of claim 11, wherein intensities of the shifted one or more annotated mass peaks of the in-silico mass spectrum are not changed from intensities of corresponding mass peaks of the experimental mass spectrum.

13. The computer program product of claim 8, wherein the experimental mass spectrum, the in-silico mass spectrum, and the unknown experimental mass spectrum are product ion spectra.

14. The computer program product of claim 8, wherein the known compound comprises a known drug of abuse and the unknown compound comprises a variant of the known drug of abuse.

15. A system for identifying an unknown compound, comprising: a processor that
obtains an experimental mass spectrum of a known compound,
annotates one or more mass peaks of the experimental mass spectrum corresponding to a substructure of the known compound with at least one modification an unknown compound is predicted to include,
creates an in-silico mass spectrum for the unknown compound from the experimental mass spectrum and the annotated one or more mass peaks,
obtains an unknown experimental mass spectrum of the unknown compound, and
determines that the unknown compound is a modification of the known compound if the unknown experimental mass spectrum matches the in-silico mass spectrum.

16. The system of claim 15 further comprising a mass spectrometer and wherein the processor instructs the mass spectrometer to analyze the unknown compound to produce the unknown experimental mass spectrum.
